(19) United States
(12) Patent Application Publication
(10) Pub. No.: US 2002/0064859 A1
(43) Pub. Date: May 30, 2002

(54) Adenovirus vectors comprising introns

(76) Inventor: Suresh K. Tikoo, Saskatoon (CA)

Correspondence Address: Gladys H. Monroy, Morrison & Foerster LLP, 755 Page Mill Road, Palo Alto, CA, 94304-1018, US

(21) Appl. No.: 09/904,782

(22) Filed: Jul. 13, 2001

Related U.S. Application Data
(60) Provisional application No. 60/218,283, filed on Jul. 14, 2000.

(51) Int. Cl. A61K 39/12; C12N 15/861; C12N 7/01

(52) U.S. Cl. 435/235.1; 424/199.1; 435/320.1

(57) ABSTRACT

The invention disclosed herein provides adenovirus vectors comprising introns 5′ to transgenes. Such adenovirus vectors are useful for expression of proteins. Methods of making and using these adenovirus vectors are provided herein. In particular, methods of obtaining increased expression of viral proteins from the adenovirus vectors comprising introns 5′ to transgenes is provided.

### CROSS-REFERENCE TO RELATED APPLICATIONS

This application claims the benefit of U.S. Provisional Application Ser. No. 60/218,283 filed Jul. 14, 2000.

### TECHNICAL FIELD

This invention relates to adenovirus, adenovirus vectors and methods of making and using adenovirus and adenovirus vectors. In particular, this invention relates to adenovirus and adenovirus vectors comprising introns 5′ to transgenes and in particular to expression of proteins in adenovirus and adenovirus vectors comprising introns 5′ to transgenes encoding said proteins.

### BACKGROUND OF THE INVENTION

In recent years, the emergence of molecular biology technology has enabled the creation of recombinant viral vectors. Proposed uses for recombinant viral vectors include delivery of toxic products to cancerous cells or infected cells, treatment of cystic fibrosis, and boosting of immune system. One type of recombinant virus vector that has received considerable attention is the adenovirus vector.

Adenoviruses are DNA viruses that have two phases of viral gene expression, early expression and late expression. Several adenovirus genes are essential for replication, such as the E1 and E2 genes for example. Other adenovirus genes such as E3, and parts of M4, are considered non-essential, that is, their deletion from an adenovirus vector does not impair replication. Adenoviruses generally undergo a lytic replication cycle following infection of a host cell. In addition to lysing the infected cell, the replicative process of adenovirus blocks the transport and translation of host cell mRNA, thus inhibiting cellular protein synthesis. For a review of adenoviruses and adenovirus replication, see Shenk, T. and Horwitz, M. S., Virology, third edition, Fields, B. N. et al., eds., Raven Press Limited, New York (1996), Chapters 67 and 68, respectively.

Adenovirus vectors are classified into two distinct groups, those that are replication-defective and those that are replication-competent. A replication-defective adenovirus vector cannot produce progeny and typically has genes essential for replication deleted. A replication-defective adenovirus vector requires a helper cell line expressing the deleted gene activity in order to replicate in a host cell. A replication-competent adenovirus vector is capable of replicating in a host cell without a helper cell line. For general background references regarding adenovirus and development of adenovirus vector systems, see Graham et al. (1973) Virology 52:456-467; Takiff et al. (1981) Lancet 11:832-834; Berkner et al. (1983) Nucleic Acid Research 11: 6003-6020; Graham (1984) EMBO J3:2917-2922; Bett et al. (1993) J Virology 67:5911-5921; Bett et al. (1994) Proc. Natl. Acad. Sci. USA 91:8802-8806; Chamberlain et al. U.S. Pat. No. 5,994,132 and He et al. U.S. Pat. No. 5,922,576.

A variety of mammalian adenoviruses are known in the art, including human adenovirus, porcine adenovirus, ovine adenovirus, canine, and bovine adenovirus. At least 47 serotypes of human adenoviruses have been described. Reviews of the most common human serotypes associated with particular diseases have been published. See for example, Foy H. M. (1989) Adenoviruses In Evans AS (ed). “Viral Infections of Humans”. New York, Plenum Publishing, pp 77-89 and Rubin B. A. (1993) Clinical picture and epidemiology of adenovirus infections, Acta Microbiol. Hung 40:303-323.

Porcine adenoviruses are disclosed in, for example, Tuboly et al 1993, Res. in Vet. Sci. 54:345-350; Derbyshire et al. 1975, J Comp. Pathol. 85:437-443; Hirahara et al. 1990, Jpn. J Vet. Sci. 52:407-409; Reddy et al. 1993, Intervirology 36:161-168; Reddy et al. 1995b, Arch. Virol. 140:195-200. Vrati et al. (1995, Virology, 209:400-408) and Xu et al. (1998, Virology 248:156-163) disclose sequences for ovine adenovirus. Morrison et al. (1997, J Gen. Virol. 78:873-878) disclose canine adenovirus type 1 DNA sequence.

The bovine adenoviruses (BAV) comprise at least ten serotypes divided into two subgroups. These subgroups have been characterized based on enzyme-linked immunoassays (ELISA), serologic studies with immunofluorescence assays, virus-neutralization tests, immunoelectron microscopy, by their host specificity and clinical syndromes. Subgroup 1 viruses include BAV 1, 2, 3 and 9 and grow relatively well in established bovine cells compared to subgroup 2 which includes BAV 4, 5, 6, 7, 8 and 10.

BAV3 was first isolated in 1965 and is the best characterized of the BAV genotypes. The nucleotide sequence of the genome (approximately 35 kb), genome organization, and transcription map of BAV3 is disclosed in Kurokawa et al 1978, J Virol. 28:212-218 and Reddy et al. (1998) J Virol 72:1394. Reddy et al. (1999, J Virol. 73: 9137) disclose a replication-defective BAV3 as an expression vector. BAV3, a representative of subgroup 1 of BAVs (Bartha 1969, Acta Vet. Acad. Sci. Hung. 19:319-321), is a common pathogen of cattle usually resulting in subclinical infection (Darbyshire et al. (1965). J Comp. Pathol. 75:327-330), though occasionally associated with a more serious respiratory tract infection (Darbyshire et al., 1966 Res. Vet Sci 7:81-93; Mattson et al., 1988, J Vet Res 49:67-69). Like other adenoviruses, BAV3 is a non-enveloped icosahedral particle of 75 nm in diameter (Niiyama et al. 1975, J Virol. 16:621-633) containing a linear double-stranded DNA molecule. BAV3 can produce tumors when injected into hamsters (Darbyshire, 1966, Nature 211:102) and bovine viral DNA can efficiently effect morphological transformation of mouse, hamster or rat cells in culture (Tsukamoto and Sugino, 1972, J. Virol. 9:465-473; Motoi et al., 1972, Gann 63:415-418). Cross hybridization was observed between BAV3 and human adenovirus type 2 (HAd2) (Hu et al., 1984, J Virol. 49:604-608) in most regions of the genome.

The use of introns in expression systems has been disclosed, see, for example, Choi T. et al. (1991, Mol Cel Biol 11 (6): 3070-4, Ill et al. WO 99/29848 and Rose et al. U.S. Pat. No. 5,861,277. Alphavirus vectors with cytomegalovirus promoter, which may include an intron is disclosed by in Parrington et al. WO 99/25858. Retroviral vectors with introns have been disclosed in Kim et al. WO 00/00629. Alphavirus-retrovirus RNA vectors which comprise genes in combination with introns or other control elements of gene expression is disclosed in Garoff et al. WO 98/15636.

The use of bovine adenovirus-3 (BAV-3) as a vector for delivery of protective antigen from pathogens of cattle is disclosed (Baxi, M. et al., 1999 Virology, 261:143-152; Reddy et al., 1999 J Virol 73, 9137-9144; Zakhartchouk et al., 1998 Virology 250, 220-229). Zakhartchouk et al., (1998 Virology 250, 220-229) disclose unsuccessful attempts to obtain high levels of expression of RNA virus genes cloned in a replication-competent BAV-3 vector without any flanking upstream or downstream sequences. Bovine coronavirus (BCV), an RNA virus, and its properties have been disclosed (King and Brian, 1982 J Virol. 42, 700-707; Deregt et al, 1989 J Gen. Virol. 70, 993-998; Deregt & Babiuk, 1987 Virology 161,410-420). Other adenovirus vectors in addition to BAV-3 have been used in attempts to improve gene expression. For example, recombinant human adenovirus-5 (HAV-5) expressing the bovine coronavirus HE gene was disclosed in Yoo et al., 1992 J Gen. Virol. 73: 2591-2600. In addition, HAV-5 expressing the bovine coronavirus HE gene was tested in cotton rats to determine the immunogenicity induced by expression of the HE gene. Baca-Estrada et al., 1995 Immunology 86, 134-140.

In spite of advances in production and use of adenovirus vectors, there exists a need for improved adenovirus vectors that enhance expression of proteins.

The disclosure of all patents and publications cited herein are hereby incorporated by reference in their entirety.

### SUMMARY OF THE INVENTION

The present invention provides adenovirus vectors comprising an intron and a heterologous transgene wherein said intron is located 5′ to the heterologous transgene and wherein said vector is capable of expressing greater levels of the heterologous transgene than a comparable adenovirus vector comprising a heterologous transgene and lacking an intron 5′ to said heterologous transgene.

In some embodiments, the adenovirus vector is a mammalian or avian adenovirus vector. In other embodiments, the mammalian adenovirus includes human, non-human primate, bovine, porcine, ovine or canine adenovirus. In other embodiments, the avian adenovirus includes turkey, chicken and other fowl. In other embodiments, the adenovirus vector is a bovine adenovirus vector (BAV), including members of subgroup 1 BAV, such as BAV 1, 2, 3 or 9, or subgroup 2 BAV, such as, BAV 4, 5, 6, 7, 8 or, 10.

In further embodiments, the adenovirus vector is replication-competent and in yet other embodiments, the adenovirus vector is replication-defective.

In additional embodiments, the transgene is a eucaryotic or prokaryotic gene including genes encoding therapeutic proteins or polypeptides; genes encoding growth hormones or other growth enhancers; and genes encoding proteins capable of eliciting an immune response. In some embodiments, the transgene encodes a protein from a pathogen, such as a viral protein, including RNA and DNA viral proteins. In other embodiments, the transgene encodes a bacterial protein or polypeptide. In yet further embodiments, the transgene encodes a protein or polypeptide from a parasite. In further embodiments, the RNA viral protein includes bovine coronavirus hemagglutinin-esterase. In yet other embodiments, the DNA viral protein includes bovine herpesvirus-1 glycoprotein D. In additional embodiments, the bacterial protein includes proteins from Haemophilis sommus, such as LppB, or Pasteurella haemolytica. In further embodiments, the parasite includes Plasmodium, associated with malaria, and members of the sub-class Coccidia, including Cryptosporidium. In further embodiments, the transgene is one which comprises a nucleic acid sequence susceptible to splicing events within a host cell.

The present invention provides host cells and compositions comprising an adenovirus vector comprising an intron and a heterologous transgene wherein said intron is located 5′ to the heterologous transgene. The present invention also provides host cells comprising recombinant adenovirus comprising an intron and a heterologous transgene wherein said intron is located 5′ to the heterologous transgene. The present invention also provides methods of making such adenovirus vectors, recombinant adenoviruses and host cells.

The present invention also provides compositions capable of inducing an immune response in a mammalian subject comprising an immunogenic composition comprising an adenovirus vector comprising an intron and a heterologous transgene wherein said intron is located 5′ to the heterologous transgene. In further embodiments, the compositions further comprise a pharmaceutically acceptable excipient. In additional embodiments, the compositions further comprise a buffer. In further embodiments, the immunogenic composition comprises a transgene encoding a protein from a pathogen. In yet further embodiments, the immunogenic compositions comprise a transgene encoding an RNA viral protein, a DNA viral protein, a bacterial protein or a protein from a parasite.

The present invention also provides methods of treating or ameliorating the symptoms of an RNA viral infection in a mammalian host comprising administering to said host a therapeutically effective amount of an immunogenic composition comprising an adenovirus vector which comprises an intron and a heterologous transgene, wherein said intron is located 5′ to the viral gene and wherein said transgene encodes an RNA viral protein. The present invention also provides methods of treating or ameliorating the symptoms of a DNA viral infection in a mammalian host comprising administering to said host a therapeutically effective amount of an immunogenic composition comprising an adenovirus vector which comprises an intron and a heterologous transgene, wherein said intron is located 5′to the viral gene and wherein said transgene encodes a DNA viral protein. The present invention also provides methods of treating or ameliorating the symptoms of a bacterial infection in a mammalian host comprising administering to said host a therapeutically effective amount of an immunogenic composition comprising an adenovirus vector which comprises an intron and a heterologous transgene, wherein said intron is located 5′to the viral gene and wherein said transgene encodes a bacterial protein. The present invention also provides methods of treating or ameliorating the symptoms of a parasitic infection in a mammalian host comprising administering to said host a therapeutically effective amount of an immunogenic composition comprising an adenovirus vector which comprises an intron and a heterologous transgene, wherein said intron is located 5′to the viral gene and wherein said transgene encodes a parasitic protein.

### BRIEF DESCRIPTION OF THE DRAWINGS

FIGS. 1A-1B. Schematic representation of recombinant E3 deleted full length BAV-3 genomic clones. FIG. 1A: BAV-3 genome ( [symbol] ), HE gene ( [symbol] ), SV40 early promoter ( [symbol] ),chimeric intron ( [symbol] ), SV40 poly(A ( [symbol] ), HCMV IE promoter ( [symbol] ). The locations of early (E) region E1, E3 and E4 are depicted. The arrow represents the direction of transcription. The name given to each recombinant virus is depicted on the right. FIG. 1B: a schematic representation of recombinant E3 deleted BAV355, which comprises bovine herpes virus type 1 glycoprotein gB (gB of BHV-1).

FIGS. 2A-2B. Restriction enzyme analysis of recombinant BAV-3 genomes. FIG. A The DNAs were extracted from BAV-3 (lane 1), BAV3.E3d (lane 2), BAV303 (lane 3), BAV332 (lane 4), BAV333 (lane 5) and BAV334 (lane 6) infected MDBK cells by Hirt's method (Hirt, 1967 J Mol. Biol. 26, 365-369) and digested with BanHI. FIG. B The fragments shown in panel A were transferred to Nytran membranes and probed with -α³²P-labeled HE probe. Lane M, 1 Kb Plus DNA ladder (Gibco/BRL) used for sizing the viral DNA fragments.

FIG. 3. Northern blot analysis of HE transcription. Total RNA was isolated from mock (M), BAV303 (lane 1,2), BAV332 (lane 3,4), BAV333 (lane 5,6) and BAV334 (lane 7,8) infected MDBK cells after 18 (1,3,5,7) or 24 (2,4,6,8) h post infection and analyzed by Northern blot analysis as described herein using 1.3 Kb Bam-HI fragment (containing HE coding sequence) of pCVE3 plasmid (Parker et al., 1989 J Gen. Virol. 70, 155-164) as a probe. Numbers on the right denote the estimated sizes of RNAs in Kb.

FIGS. 4A-4D. Expression of HE protein in MDBK cells infected with recombinant BAV3 viruses. Proteins from lysates of radiolabeled mock infected (lane 1) BAV-3 infected (lane 2), BCV infected (lane 3) BAV303 infected (FIG. 4A, lanes 4-6), BAV332 infected (FIG. 4B, lanes 4-6), BAV333 infected (FIG. 4C, lanes 4-6) or BAV334 infected (FIG. 4D, lanes 4-6) MDBK cells were immunoprecipitated with polyclonal anti-BCV serum and analyzed by SDS-PAGE under reducing conditions. The cells were harvested at 12 (lane 4), 24 (lane 5) and 36 h (lane 6) post-infection. The position of the size markers (in kilodaltons) are shown to the left of each panel.

FIGS. 5A-5B. Restriction enzyme analysis of recombinant BAV-3 genomes. FIG.5A. The DNAs were extracted from the BAV-3 (lane 1), BAV3.E3 d (lane 2) and BAV335 (lane 3) infected MDBK cells by Hirt's method (Hirt, 1967 J Mol. Biol. 26, 365-369) and digested with BamHI. FIG. 5B. The fragments shown in FIG. 5A were transferred to Nytran membranes and probed with a³²P-labeled gB probe. Lane M, 1 kb DNA Plus ladder (Gibco/BRL) used for sizing of the DNA fragments.

FIG. 6. Expression of gB protein in MDBK cells. Proteins from lysates of radiolabeled mock infected (lane 1), BAV-3 infected (lane 2), BHV-1 infected (lane 3) or BAV335 infected MDBK cells harvested at 12 (lane 4), 24 (lane 5) and 36 h (lane 6) post-infection were immunoprecipitated with a pool of BHV-1 gB specific monoclonal antibodies and analysed by SDS-PAGE under reducing conditions. Positions of the size markers (in kilodaltons) are shown to the left of the panel.

### MODES FOR CARRYING OUT THE INVENTION

The present inventors have constructed adenovirus vectors comprising introns located 5′, that is, upstream from transgenes and have demonstrated increased expression of transgenes in adenovirus vectors comprising an intron located 5′to a transgene when compared to a comparable adenovirus vector comprising the transgene and lacking an intron located 5′to the transgene. In some embodiments of the present invention exemplified herein, the transgene encodes a protein from a pathogen. In other embodiments, the transgene encodes an RNA viral protein, a DNA viral protein, a bacterial protein or a protein from a parasite.

In some embodiments, the transgene is one which comprises a nucleic acid sequence susceptible to splicing events within a host cell due to the inherent characteristics of the sequence. The presence of a nucleic acid sequence susceptible to splicing events within a host cell in transgene sequences may contribute to problems associated with expression of incomplete or truncated or otherwise aberrant transgene sequences. The presence of an intron located 5′to the transgene sequence comprising a nucleic acid sequence susceptible to splicing events within a host cell is associated with increased expression of the transgene.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); The Immunoassay Handbook (David Wild, ed., Stockton Press NY, 1994); Antibodies: A Laboratory Manual (Harlow et al., eds., 1987), Virology, 3rd ed. (Fields et al., ed. Chanock et al., pub Lippincott and Raven, Philadelphia), and Methods of Immunological Analysis (R. Masseyeff, W. H. Albert, and N. A. Staines, eds., Weinheim: VCH Verlags gesellschaft mbH, 1993). For techniques related to adenovirus, see, inter alia, Felgner and Ringold (1989) Nature 337:387-388; Berkner and Sharp (1983) Nucl. Acids Res. 11:6003-6020; Graham (1984) EMBO J. 3:2917-2922; Bett et al. (1993) J Virology 67:5911-5921; Bett et al. (1994) Proc. Natl. Acad. Sci. USA 91:8802-8806.

Definitions

As used herein, the term “intron” refers to non-coding DNA or precursor RNA sequences which are not present in mature RNA and encompasses naturally occurring isolated introns as well as synthetic introns and chimeric introns, that is, introns which comprise portions from different nucleic acid species. Introns encompassed within the invention include introns of eucaryotic origin. Introns are generally spliced out of messenger RNA when transcription occurs and generally contain splice donor and splice acceptor sites. Introns as used herein encompass a 5′splice donor site and a 3′ splice acceptor site. As used herein a “splice donor site” refers to sequences at the 5′end of an intron, that is, at the 5′junction of the exon-intron. As used herein a “splice acceptor site” refers to sequences at the 3′ end of an intron, that is, at the 3′ junction of the intron-exon. Splice donor and acceptor sites are disclosed in Mount S., 1982, Nucleic Acids Research, 10:459-471. In the adenovirus vector constructs of the present invention, it is preferred that the intron be located 3′ to the transgene promoter and 5′to the beginning codon of the transgene.

An “adenovirus vector” or “adenoviral vector” (used interchangeably) comprises a polynucleotide construct of the invention. A polynucleotide construct of this invention may be in any of several forms, including, but not limited to, DNA, DNA encapsulated in an adenovirus coat, DNA packaged in another viral or viral-like form (such as herpes simplex, and AAV), DNA encapsulated in liposomes, DNA complexed with polylysine, complexed with synthetic polycationic molecules, conjugated with transferrin, and complexed with compounds such as PEG to immunologically “mask” the molecule and/or increase half-life, and conjugated to a nonviral protein. Preferably, the polynucleotide is DNA. As used herein, “DNA” includes not only bases A, T, C, and G, but also includes any of their analogs or modifi ed forms of these bases, such as methylated nucleotides, internucleotide modifications such as uncharged linkages and thioates, use of sugar analogs, and modified and/or alternative backbone structures, such as polyamides. The term adenovirus vector encompasses those that are replication-competent and those that are replication-defective in a target cell. As used herein, the term “comparable” when comparing adenovirus vectors comprising introns to adenovirus vectors lacking introns, it is meant that the adenovirus vectors are identical or essentially identical except for the presence of the intron.

The term “transgene” refers to a heterologous gene that is inserted into an adenovirus genome or vector to create an adenovirus construct. Transgenes can be inserted by recombination or by restriction enzyme cutting and pasting into the adenovirus genome with DNA ligase. The term “upstream” in reference to a gene or element means that the position is 5′to the reference point. For example, if element A is upstream of gene B, then reading the genomic sequence 5′to 3′, element A would be 5′to gene B. The term “downstream” in reference to a gene or element means that the position is 3′ to the reference point. For example, if element A is downstream of gene B, then reading the genomic sequence 5′to 3′, element A would be 3′ to gene B. A transgene may contain a nucleic acid sequence susceptible to splicing events within a host cell due to the inherent characteristics of the sequence. Examples of such transgenes include, but are not limited to, RNA viral genes. Transgenes encompassed within the invention include eucaryotic and prokaryotic genes, including genes encoding therapeutic proteins or polypeptides; genes encoding growth hormones or other growth enhancers; and genes encoding proteins capable of eliciting an immune response. In some embodiments, the transgene encodes a viral protein such as an RNA or DNA viral protein. In other embodiments, the transgene encodes a bacterial gene.

As used herein, the term “vector” refers to a polynucleotide construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, or a “viral vector” which is designed to result in the production of a recombinant virus or virus-like particle, or “shuttle vectors”, which comprise the attributes of more than one type of vector.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxynucleoside phosphoramidate (P-NH2) or a mixed phosphoramidate-phosphodiester oligomer. Peyrottes et al. (1996) Nucleic Acids Res. 24: 1841-8; Chaturvedi et al. (1996) Nucleic Acids Res. 24: 2318-23; Schultz et al. (1996) Nucleic Acids Res. 24: 2966-73. A phosphorothioate linkage can be used in place of a phosphodiester linkage. Braun et al. (1988) J Immunol. 141: 2084-9; Latimer et al. (1995) Molec. Immunol. 32: 1057-1064. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A polynucleotide or polynucleotide region has a certain percentage (for example, 80%, 85%, 90%, or 95%) of “sequence identity” to another sequence means that, when aligned, that percentage of bases are the same in comparing the two sequences. This alignment and the percent homology or sequence identity can be determined using software programs known in the art, for example those described in Current Protocols in Molecular Biology (F. M. Ausubel et al., eds., 1987) Supplement 30, section 7.7.18. A preferred alignment program is ALIGN Plus (Scientific and Educational Software, Pennsylvania), preferably using default parameters, which are as follows: mismatch=2; open gap=0; extend gap=2.

“Under transcriptional control” is a term well understood in the art and indicates that transcription of a polynucleotide sequence, usually a DNA sequence, depends on its being operably (operatively) linked to an element which contributes to the initiation of, or promotes, transcription. “Operably linked” refers to a juxtaposition wherein the elements are in an arrangement allowing them to function.

A “heterologous” region of a DNA construct is an identifiable segment of DNA within or attached to another DNA molecule that is not found in association with the other molecule in nature. Thus, when the heterologous region encodes a viral gene, the gene will usually be flanked by DNA that does not flank the viral gene in the genome of the source virus or virus-infected cells. Another example of the heterologous coding sequence is a construct wherein the coding sequence itself is not found in nature (e.g., synthetic sequences having codons different from the native gene). Allelic variation or naturally occurring mutational events do not give rise to a heterologous region of DNA, as used herein.

The term “heterologous gene” or “heterologous transgene” is any gene that is not present in wild-type adenovirus. Preferably, the heterologous gene will not be expressed by the adenovirus or adenovirus vector prior to the introduction of the heterologous gene into the adenovirus or adenovirus vector. For the present invention, a heterologous region that is inserted into an adenovirus vector is not comprised of nucleotide sequences normally found in an adenovirus genome. It is understood that a small degree of sequence homology may occur between the heterologous region and the adenovirus vector, particularly if the heterologous region is derived from a virus related to the adenovirus or in the same family as the adenovirus.

A “double-stranded DNA molecule” refers to the polymeric form of deoxyribonucleotides (adenine, guanine, thymine, or cytosine) in its normal, double-stranded helix. This term refers only to the primary and secondary structure of the molecule, and does not limit it to any particular tertiary forms. Thus, this term includes double-stranded DNA found, inter alia, in linear DNA molecules (e.g., restriction fragments of DNA from viruses, plasmids, and chromosomes). In discussing the structure of particular double-stranded DNA molecules, sequences may be described herein according to the normal convention of giving only the sequence in the 5′to 3′ direction along the nontranscribed strand of DNA (i.e., the strand having the sequence homologous to the mRNA).

A DNA “coding sequence” is a DNA sequence which is transcribed and translated into a polypeptide in vivo when placed under the control of appropriate regulatory sequences. The boundaries of the coding sequence are determined by a start codon at the 5′(amino) terminus and a translation stop codon at the 3′ (carboxy) terminus. A coding sequence can include, but is not limited to, procaryotic sequences, cDNA from eucaryotic mRNA, genomic DNA sequences from eucaryotic (e.g., mammalian) DNA, viral DNA, and even synthetic DNA sequences. A polyadenylation signal and transcription termination sequence will usually be located 3′ to the coding sequence.

A “transcriptional promoter sequence” is a DNA regulatory region capable of binding RNA polymerase in a cell and initiating transcription of a downstream (3′ direction) coding sequence. For purposes of defining the present invention, the promoter sequence is bound at the 3′ terminus by the translation start codon (ATG) of a coding sequence and extends upstream (5′direction) to include the minimum number of bases or elements necessary to initiate transcription at levels detectable above background. Within the promoter sequence will be found a transcription initiation site (defined by mapping with nuclease S1), as well as protein binding domains (consensus sequences) responsible for the binding of RNA polymerase. Eucaryotic promoters will often, but not always, contain “TATA” boxes and “CAAT” boxes. Procaryotic promoters contain Shine-Dalgamo sequences in addition to the −10 and −35 consensus sequences.

DNA “control sequences” refer collectively to promoter sequences, ribosome binding sites, splicing signals, polyadenylation signals, transcription termination sequences, upstream regulatory domains, enhancers, translational termination sequences and the like, which collectively provide for the transcription and translation of a coding sequence in a host cell.

A coding sequence or sequence encoding is “operably linked to” or “under the control of” control sequences in a cell when RNA polymerase will bind the promoter sequence and transcribe the coding sequence into mRNA, which is then translated into the polypeptide encoded by the coding sequence.

A “clone” is a population of daughter cells derived from a single cell or common ancestor. A “cell line” is a clone of a primary cell that is capable of stable growth in vitro for many generations.

“Bovine host” refers to cattle of any breed, adult or infant.

The term “protein” is used herein to designate a polypeptide or glycosylated polypeptide, respectively, unless otherwise noted. The term “polypeptide” is used in its broadest sense, i.e., any polymer of amino acids (dipeptide or greater) linked through peptide bonds. Thus, the term “polypeptide” includes proteins, oligopeptides, protein fragments, analogs, muteins, fusion proteins and the like.

“Native” proteins or polypeptides refer to proteins or polypeptides recovered from adenovirus or adenovirus-infected cells. Thus, the term “native BAV polypeptide” would include naturally occurring BAV proteins and fragments thereof. “Non-native” polypeptides refer to polypeptides that have been produced by recombinant DNA methods or by direct synthesis. “Recombinant” polypeptides refers to polypeptides produced by recombinant DNA techniques; i.e., produced from cells transformed by an exogenous DNA construct encoding the desired polypeptide.

A “substantially pure” protein will be free of other proteins, preferably at least 10% homogeneous, more preferably 60% homogeneous, and most preferably 95% homogeneous.

An “antigen” refers to a molecule containing one or more epitopes that will stimulate a host's immune system to make a humoral and/or cellular antigen-specific response. The term is also used interchangeably with “immunogen.”

An “immunological response” or “immune response” is the induction and/or development in the host of a cellular and/or humoral immune response to the composition being administered. The composition can be a vaccine or alternatively a virus or viral vector. In general, cellular immune responses consist of the host generating cytokines (interferons, TNF, interleukin, etc.), or chemokines, or displaying cytotoxic killing by cytotoxic T cells. In addition, helper T cells and suppressor T cells can also be involved in the cellular immune response. Humoral immune response generally consists of B cells producing antibodies directed to an antigen(s) or epitope(s) included in the composition or vaccine of interest. Also included in immune responses are innate immune responses by natural killer cells, macrophages, neutrophils, eosinophils, and basophils.

The term “mammalian” encompasses any mammalian species including humans, non-human primates, rodents, dogs, cats, rabbits, pigs, bovines, and sheep.

A “host cell” is a cell which has been transformed, or is capable of transformation, by an exogenous DNA sequence.

A cell has been “transformed” by exogenous DNA when such exogenous DNA has been introduced inside the cell membrane. Exogenous DNA may or may not be integrated (covalently linked) to chromosomal DNA making up the genome of the cell. In procaryotes and yeasts, for example, the exogenous DNA may be maintained on an episomal element, such as a plasmid. A stably transformed cell is one in which the exogenous DNA has become integrated into the chromosome so that it is inherited by daughter cells through chromosome replication. For mammalian cells, this stability is demonstrated by the ability of the cell to establish cell lines or clones comprised of a population of daughter cell containing the exogenous DNA.

“Replication” and “propagation” are used interchangeably and refer to the ability of an adenovirus vector of the invention to reproduce or proliferate. These terms are well understood in the art. For purposes of this invention, replication involves production of adenovirus proteins and is generally directed to reproduction of adenovirus. Replication can be measured using assays standard in the art, such as a burst assay or plaque assay. “Replication” and “propagation” include any activity directly or indirectly involved in the process of virus manufacture, including, but not limited to, viral gene expression; production of viral proteins, nucleic acids or other components; packaging of viral components into complete viruses; and cell lysis.

As used herein, “replication-competent” adenovirus refers to an adenovirus that can produce progeny, that is, that can replicate in a host cell. Replication can be measured using assays standards in the art, such as burst assay, plaque assay, or a one-step growth assay.

As used herein, “replication-defective” adenovirus refers to adenovirus that cannot produce progeny, that is, that cannot replicate in a host cell. Typically, a gene(s) essential for replication has been deleted from the host cell. Replication-defective adenovirus can be grown in the presence of helper cell lines (i.e., 293 cells for HAV or R2 cells for BAV (Reddy et al., 1999, J Virol 73: 9137-9144 and ATCC deposit PTA-156) that supply the necessary genes essential for replication.

The term “insertion site” refers to a location within the adenovirus genome wherein a transgene is inserted. The insertion site can be a region of homologous sequences in the adenovirus or adenovirus vector such that a transgene is inserted by homologous recombination. The insertion site can alternatively be a site for restriction enzyme cleavage wherein a transgene is inserted into the cleaved site and then ligated together into an adenovirus construct by using DNA ligase.

“A,” “an” and “the” include plural references unless the context clearly dictates otherwise.

General Methods

I. Adenovirus vectors

Mammalian adenovirus vectors have been disclosed, see, for example, Shenk, T. and Horwitz, M. S., Virology, third edition, Fields, B. N. et al., eds., Raven Press Limited, New York (1996), Chapters 67 and 68, respectively; Graham et al. (1973) Virology 52:456-467; Takiffetal. (1981) Lancet 11:832-834; Berkneret al. (1983) Nucleic Acid Research 11: 6003-6020; Graham (1984) EMBO J 3:2917-2922; Bett et al. (1993) J Virology 67:5911-5921; Bett et al. (1994) Proc. Natl. Acad. Sci. USA 91:8802-8806; and Chamberlain et al. U.S. Pat. No. 5,994,132. There are several mammalian forms of adenovirus vectors, including human, porcine, ovine, canine, and bovine adenoviruses from which a skilled artisan may choose for purposes of practicing this invention.

Human adenoviruses Ad3, Ad4, Ad5, Ad9 and Ad35 are available from the American Tissue Culture Collection ATCC). The National Center for Biotechnology Information GenBank accession number for Ad5 is M73260/M29978; for Ad9 X74659; and for Ad35, U10272. Chow et al. (1977, Cell 12:1-8) disclose human adenovirus 2 sequences; Davison et al. (1993, J. Mole. Biol. 234:1308-1316) disclose the DNA sequence of human adenovirus type 40; Sprengel et al. (1994, J Virol. 68:379-389) disclose the DNA sequence for human adenovirus type 12 DNA; Vrati et al. (1995, Virology, 209:400-408) disclose sequences for ovine adenovirus; Morrison et al. (1997, J. Gen. Virol 78:873-878) disclose canine adenovirus type 1 DNA sequence; and Reddy et al. (1998, Virology, 251:414) disclose DNA sequences for porcine adenovirus.

In some embodiments of this invention, the adenovirus vector is constructed with bovine adenovirus, such as bovine adenovirus-3 (BAV3). Reddy et al. (1998) Journal of Virology 72:1394 disclose nucleotide sequences for BAV3. Although the size (34,446 bp) and the overall organization of the BAV3 genome appear to be similar to that of HAVs, there are certain differences. Reddy et al. (1998) supra. One of the distinctive features of the BAV3 genome is the relatively small size of the E3 coding region (1517 bp). Mittal et al. (1992) J. Gen. Virol. 73:3295-3300; Mittal et al. (1993). J Gen. Virol. 74:2825; and Reddy et al. (1998) supra. Analysis of the sequence of the BAV3 E3 region and its RNA transcripts suggests that BAV3 E3 may encode at least four proteins, one of which (121R) exhibits limited homology with the 14.7 kDa protein of HAV5. Idamakanti (1998) “Molecular characterization of E3 region of bovine adenovirus-3,” M.Sc. thesis, University of Saskatchewan, Saskatoon, Saskatchewan.

Adenovirus vector constructs can then undergo recombination in vitro or in vivo, with a BAV genome either before or after transformation or transfection of an appropriate host cell.

Suitable host cells include any cell that will support recombination between a BAV genome and a plasmid containing BAV sequences, or between two or more plasmids, each containing BAV sequences. Recombination is generally performed in procaryotic cells, such as E. coli, while transfection of a plasmid containing a viral genome, to generate virus particles, is conducted in eukaryotic cells, preferably mammalian cells, more preferably bovine cell cultures, most preferably MDBK or PFBR cells, and their equivalents. The growth of bacterial cell cultures, as well as culture and maintenance of eukaryotic cells and mammalian cell lines are procedures which are well-known to those of skill in the art.

II. Introns

The invention encompasses introns of eucaryotic origin and introns having a variety of properties as long as the adenovirus vector comprising the intron 5′to the desired transgene exhibits increased expression of the transgene as compared to the transgene expression in a comparable adenovirus vector comprising the transgene and lacking an intron. Measurement of expression levels of transgenes is deemed routine for one of skill in the art. In a preferred embodiment, the intron is located 3′ to the promoter of the transgene, that is, in a preferred embodiment, the order within the adenovirus vector is promoter-intron-transgene optionally followed by a poly A sequence. Introns encompassed within the invention comprise those having a variety of splice donor/acceptor site properties, and may be of any size. Generally speaking, introns will be between about 30 base pairs (bp) to about 2 kb in size, about 100 bp to about 1 kb in size, and about 200 bp to about 700 bp in size and any eucaryotic intron may be used as long as the adenovirus vector comprising the intron 5′to the desired transgene exhibits increased expression of the transgene as compared to the transgene expression in a comparable adenovirus vector comprising the transgene and lacking an intron. In embodiments disclosed herein, introns comprising strong donor/acceptor sites are used in constructs when the transgene comprises a nucleic acid sequence susceptible to splicing events within a host cell due to the inherent characteristics of the sequence, such as an RNA viral gene comprising such a nucleic acid sequence. A catalogue of splice junction sequences including donor sites (also referred to as exon-intron boundary sequence) and acceptor sites (also referred to as intron-exon boundary sequences) is provided in Mount (1982, Nucleic Acid Research vol. 10: 459-472) incorporated herein in its entirety. Mount et al. describe the consensus sequence of exon-intron boundaries (splice donor) as

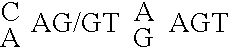

and the consensus sequence of intron-exon boundaries (splice acceptor)as:

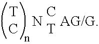

Transgenes comprising nucleic acid sequences susceptible to splicing events within a host cell can be determined by comparing the transgene sequence to the splice junction sequences provided in Mount, et al. supra. A transgene comprising a nucleic acid sequence having a high degree of sequence identity, e.g. for example, 85%, 90% or 95% or greater sequence identity, as measured by ALIGN Plus (Scientific and Educational Software, Pennsylvania), preferably using default parameters, which are as follows: mismatch=2; open gap=0; extend gap=2, to a splice junction sequence disclosed in Mount et al. may be susceptible to splicing events with a host cell.

The present invention encompasses introns from eucaryotic species heterologous to the adenovirus species, introns from eucaryotic species homologous to the adenovirus species, and introns from species heterologous or homologous to the transgene sequence. For example, an intron from a human genomic sequence is used in an adenovirus construct comprising a heterologous transgene encoding an RNA or DNA viral protein, bacterial protein or protein from a parasite. In another example, an intron from a human genomic sequence is used with a nonhuman mammalian adenovirus vector. The present invention encompasses introns isolated from naturally occurring sources as well as synthetic or chimeric introns, that is hybrid introns constructed from two species or different intron portions, such as donor and acceptor sites, from the same species. In an embodiment exemplified herein, the chimeric intron described in Senapathy et al. 1990 Meth. Enzymol. 183: 252-278, which comprises the 5′-donor site from the first intron of the human beta-globulin gene and the branch and 3′-acceptor site from the intron of an immunoglobulin gene heavy chain variable region, is used in a adenovirus construct comprising an RNA viral protein.

In embodiments wherein the transgene is thought to contain a nucleic acid sequence susceptible to splicing events, such as an RNA viral protein, it is preferred to use an intron having a strong splice acceptor and/or splice donor site. Without wanted to be bound by theory, the rationale behind the need for strong splice acceptor and donor sites for adenovirus vectors comprising RNA viral proteins is that the nucleic acid sequences encoding RNA viral proteins may contain nucleic acid sequences susceptible to splicing events because RNA viral sequences are transcribed in the cytoplasm, where there is no cell splicing machinery. The existence of nucleic acid sequences susceptible to splicing events in heterologous transgenes, in particular, when the heterologous transgene is a RNA viral protein, may affect the overall protein expression of the protein should the cellular splicing machinery recognize and use the sequence susceptible to splicing events. The strength of a putative splice donor and/or acceptor site can be determined by comparing the sequences comprising the putative splice donor and/or acceptor site to the sequences provided by Mount, supra. Also, methods of assaying the strength of a splice donor/acceptor sites include, but are not limited to, RT-PCR of the transcripts produced with subsequent gel electrophoresis to determine size.

Addition of introns into the adenovirus vector is achieved by adding a 5′splice donor site and a 3′ splice acceptor site 5′to the desired transgene and preferably 3′ to the transgene promoter sequence. Addition of an intron into an adenovirus vector can be effected either prior to the addition of the heterologous transgene, after the addition of the heterologous transgene or simultaneously through design of the vector construct. If the intron is being added to remove a portion of the adenovirus genome, then the 5′ splice donor site is placed at the 5′ end of the adenovirus portion being removed and the 3′ splice acceptor site is placed at the 3′ end of the adenovirus portion being removed. Preferably, the cellular splicing machinery will readily select the introduced intron 5′ splice donor sites and 3′ splice acceptor sites over any other surrounding nucleic acid sequences susceptible to splicing events, such as those occurring in the transgene.

III. Transgenes sequences

Transgenes encompassed within the present invention include but are not limited to eucaryotic and prokaryotic genes, including genes encoding therapeutic proteins or polypeptides; genes encoding growth hormones or other growth enhancers; and genes encoding proteins capable of eliciting an immune response, such as antigens from pathogenic organisms. Transgenes encoding desired antigens, or antigenic fragments thereof, include those of organisms which cause disease in mammals, particularly bovine pathogens such as bovine rotavirus (RNA virus), bovine coronavirus (RNA virus), bovine herpes virus type 1 (DNA virus), bovine respiratory syncytial virus (RNA virus), bovine parainfluenza virus type 3 (BPI-3) (RNA virus), bovine diarrhea virus (RNA virus), Pasteurella haemolytica, Haemophilus somnus and the like. In some embodiments, the transgene encodes a protein from a pathogen. In other embodiments, the transgene encodes a viral protein such as an RNA or DNA viral protein. In other embodiments, the transgene encodes a bacterial gene, such as LppB. In further embodiments, the transgene encodes a protein from a parasite, such as a protein from a member of Coccidia.

In embodiments illustrated herein, the transgene is a viral protein, such as an RNA viral protein or DNA viral protein. Since viral proteins are potential antigens, the quality of immune response in animals immunized with recombinant adenoviruses comprising viral proteins may be associated with the levels of expression of the viral protein, ie, the potential antigen. In addition, since the development of a quality immune response is likely to correlate with levels of antigen produced by the recombinant adenovirus vectors, methods of effectively increasing RNA viral gene expression in adenovirus expression system are highly desirable. For example, the expression of a bovine antigen, a glycoprotein of bovine coronavirus (BCV), is desirable since BCV infection causes neonatal diarrhea in calves. The resulting ramifications of BCV infection are significant economic losses due to mortality and decreased productivity of the survivors. Since currently available vaccines against BCV are not effective (Waltner-Toews et al., 1985 Can. J Comp. Med. 49,1-9), better vaccines are needed to reduce the economic losses.

The bovine coronavirus (BCV) contains positive single stranded RNA genome of about 30 kb in length. The BCV genome encodes three membrane glycoproteins, the integral membrane protein (M), the spike protein (S), and the haemagglutinin-esterase (HE) (King and Brian, 1982 J Virol. 42, 700-707). The S and HE proteins are major membrane associated glycoproteins and induce virus neutralizing antibodies (Deregt et al., 1989 J Gen. Virol. 70, 993-998). The monoclonal antibodies raised against the HE protein neutralized the infectivity of BCV under cell culture conditions and protected the intestinal epithelia of cattle from the virus infection (Deregt & Babiuk, 1987 Virology 161, 410-420).

Adenovirus vectors of this invention comprise an intron upstream of a transgene and preferably downstream of the transgene promoter. In the case where adenovirus vectors are being used for vaccination, the selection of transgene becomes important to elicit an immune response to the heterologous protein.

If the end result desired is an immune response to the transgene, as is the case with many adenovirus vector designed for vaccination purposes, transgene sequences should be selected that code for immunogenic, rather than non-immunogenic, proteins and inserted into the adenovirus genome at a desired insertion site. Selection of the immunogenic viral protein can be determined by obtaining antibody from the host infected with the virus and then using the antibody in binding assays to various in vitro translated proteins of that virus to determine specificity. See for example, Khattar, 1995, Virology 213:28-37 and Idamakanti et al., 1999, Virology, 265: 351-359. Once the specific protein is identified to which the host's antibody binds, the nucleotide sequence of that specific protein can be used as the transgene for insertion into the adenovirus vector. The adenovirus vector is then administered to a host in a pharmaceutically acceptable excipient and subsequent immune responses are then monitored.

In other embodiments, the heterologous transgene encodes a bacterial protein. Methods for isolating bacterial sequences are well-known in the art. In one embodiment, the bacterium is Haemophilis sommus, and the protein is LppB. In another embodiment, the bacterium is Pasteurella haemolytica. One aspect to consider in selecting a bacterial sequence for use as a heterologous transgene is the immunogenicity of the bacterial protein. Bacterial proteins can be tested for their ability to induce an immune response in the host mammal by means known to those of skill in the art.

In yet further embodiments, the transgene encodes a protein from a parasite. Parasitic organisms are described in for example, Medical Microbiology & Immunology 3rd Edition authors, Levinson, et al., publ. Appleton & Lange, Conneticut, see in particular Chap 6 at page 249. Parasites include for example, Entamoeba, Giardia, Cryptosporidium, Trichomonas, Trypanosoma, Leishmania, Plasmodium, Toxoplasma, Pnuemocystis and members of the sub-class Coccidiae. One aspect to consider in selecting a parasitic sequence for use as a heterologous transgene is the immunogenicity of the protein encoded. Proteins from parasites can be tested for their ability to induce an immune response in the host mammal by means known to those of skill in the art.

Immune responses to the transgene that is expressed can be monitored by any number of methods known in the art. Humoral response is typically monitored by measuring antibodies titers to the transgene. The antibodies can either be measured in serum or purified from the serum before subjecting to any tests. Antibody titers can be determined by any number of methods including, but not limited to, ELISA, ELISPOT, PCR, and flow cytometry. These methods also allow for specificity to the transgene to be determined. Cellular immune response can be determined by any number of methods known in the art including, but not limited to, cytotoxicity assays with CTL or NK cells, ELISA to cytokines or chemokines, RT-PCR to detect message from transcription of cytokines or chemokines, flow cytometry, or proliferation assays.

The level of expression or activity of the transgene can be controlled by several factors. The kinetics of expression can change depending on the type of promoter used. For example, as demonstrated herein, inclusion of simian virus (SV)-40 promoter in one of the vectors of this invention causes earlier expression of the transgene than when the human cytomegalovirus (HCMV) IE promoter is used. Yet another factor to consider when selecting a transgene is the size of the transgene. Smaller genes may be easier to transcribe and translate into proteins and therefore, more protein is expressed at the end of a defined time period.

In some embodiments of the adenovirus vector, the adenovirus vector comprises the chloramphenicol acetyltransferase (CAT) gene which can be used as a marker for determining activity. In other embodiments of the adenovirus vector, the vector comprises a putative target for phosphorylation. Measurement of transgene activity or expression is accomplished by any of the methods known in the art including, but not limited to, protein assays (i.e. Lowrey assay, Bradford assays, etc.) using readily available kits (i.e. Biorad), gel electrophoresis, CAT assays, and phosphorylation assays.

In another aspect of this invention, host cells comprising adenovirus vectors comprising introns upstream of transgenes are provided. Introduction of an adenovirus vector comprising an intron upstream of a heterologous transgene into cells can be achieved by any method known in the art, including, but not limited to, microinjection, transfection, infection, electroporation, CaPO₄ precipitation, DEAE-dextran, liposomes, and particle bombardment. In a preferred embodiment, the host cells are eucaryotic cells which can provide cellular splicing machinery. In another embodiment, the adenovirus vectors are packaged as infectious adenovirus that can infect host cells. A criteria to use in the selection of a host cell is the requirement that the host cell be able to support the growth and replication of the adenovirus vector or adenovirus. An additional requirement is that the host cell needs to be able to support the expression of the heterologous transgene.

In another aspect of this invention, compositions comprising an adenovirus vector described herein and kits comprising an adenovirus vector described are also provided. In one embodiment, the composition comprising an adenovirus vector described herein further comprises a pharmaceutically acceptable excipient. The adenovirus vector can be packaged in an adenovirus in a physiologically buffered solution, such a media or phosphate buffered saline (PBS).

IV. Construction of adenovirus vectors

Description of construction of adenovirus vectors is exemplified with BAV but applies similarly to any mammalian species of adenovirus vector.

One or more heterologous sequences can be inserted into one or more regions of the mammalian adenovirus genome, such as the BAV genome, to generate a recombinant adenovirus vector, limited only by the insertion capacity of the genome and ability of the recombinant vector to express the inserted heterologous sequences. In general, adenovirus genomes can accept inserts of approximately 5% of genome length and remain capable of being packaged into virus particles. The size of insertion of foreign genetic material is thought to be limited to about 1.8 kb to 2 kb. If the foreign genetic sequence to be inserted is greater than 2 kb, deletion of an adenovirus gene(s) allows more foreign sequence to be packaged. The insertion capacity can be increased by deletion of non-essential regions and/or deletion of essential regions whose function is provided by a helper cell line.

In some embodiments of the invention wherein the adenovirus genome is the BAV genome, insertion can be achieved by constructing a plasmid containing the region of the BAV genome into which insertion of a heterologous transgene is desired. In some embodiments of the invention, the heterologous transgene encodes an RNA viral protein, a DNA viral protein, a bacterial protein, a parasitic protein or the transgene is one which comprises a nucleic acid sequence susceptible to splicing events within a host cell and an intron is placed 5′ to the transgene and 3′ to the transgene promoter. The promoter may be the naturally occurring promoter for the transgene or a heterologous promoter. In the alternative, a desired therapeutic protein can be inserted into the BAV. The plasmid is then digested with a restriction enzyme having a recognition sequence in the BAV portion of the plasmid, and a heterologous sequence is inserted at the site of restriction digestion. The plasmid, containing a portion of the BAV genome with an inserted heterologous sequence, is co-transformed, along with a BAV genome or a linearized plasmid containing a BAV genome, into a bacterial cell (such as, for example, E. coli), wherein the BAV genome can be a full-length genome or can contain one or more deletions. Homologous recombination between the plasmids generates a recombinant BAV genome containing inserted heterologous sequences. See He et al U.S. Pat. No. 5,922,576.

Deletion of BAV sequences, to provide a site for insertion of heterologous sequences or to provide additional capacity for insertion at a different site, can be accomplished by methods well-known to those of skill in the art. For example, for BAV sequences cloned in a plasmid, digestion with one or more restriction enzymes (with at least one recognition sequence in the BAV insert) followed by ligation will, in some cases, result in deletion of sequences between the restriction enzyme recognition sites. Alternatively, digestion at a single restriction enzyme recognition site within the BAV insert, followed by exonuclease treatment, followed by ligation will result in deletion of BAV sequences adjacent to the restriction site. A plasmid containing one or more portions of the BAV genome with one or more deletions, constructed as described above, can be co-transfected into a bacterial cell along with a BAV genome (full-length or deleted) or a plasmid containing either a full-length or a deleted BAV genome to generate, by homologous recombination, a plasmid containing a recombinant BAV genome with a deletion at one or more specific sites. BAV virions containing the deletion can then be obtained by transfection of mammalian cells (including, but not limited to, MDBK or PFBR cells and their equivalents) with the plasmid containing the recombinant BAV genome.

In one embodiment of the invention, insertion sites are adjacent to and downstream (in the transcriptional sense) of BAV promoters. Locations of BAV promoters, and restriction enzyme recognition sequences downstream of BAV promoters, for use as insertion sites, can be easily determined by one of skill in the art from the BAV nucleotide sequence. Alternatively, various in vitro techniques can be used for insertion of a restriction enzyme recognition sequence at a particular site, or for insertion of heterologous sequences at a site that does not contain a restriction enzyme recognition sequence. Such methods include, but are not limited to, oligonucleotide-mediated heteroduplex formation for insertion of one or more restriction enzyme recognition sequences (see, for example, Zoller et al. (1982) Nucleic Acids Res. 10:6487-6500; Brennan et al. (1990) Roux's Arch. Dev. Biol 199:89-96; and Kunkel et al. (1987) Meth. Enzymology 154:367-382) and PCR-mediated methods for insertion of longer sequences. See, for example, Zheng et al. (1994) Virus Research 31:163-186.

It is also possible to obtain expression of a heterologous sequence inserted at a site that is not downstream from a BAV promoter, if the heterologous sequence additionally comprises transcriptional regulatory sequences that are active in eukaryotic cells. Such transcriptional regulatory sequences can include cellular promoters such as, for example, the bovine hsp70 promoter and viral promoters such as, for example, herpesvirus, adenovirus and papovavirus promoters and DNA copies of retroviral long terminal repeat (LTR) sequences.

In another embodiment, homologous recombination in a procaryotic cell can be used to generate a cloned BAV genome; and the cloned BAV genome can be propagated as a plasmid. See for example, U.S. Pat. No. 5,922,576. Infectious virus can be obtained by transfection of mammalian cells with the cloned BAV genome rescued from plasmid-containing cells.

Adenovirus vectors expressing foreign genes have described in various publications. For techniques related to adenovirus, see, inter alia, Felgner and Ringold (1989) Nature 337:387-388; Berkner and Sharp (1983) Nucl. Acids Res. 11:6003-6020; Graham (1984) EMBO J. 3:2917-2922; Bett et al. (1993) J Virology 67:5911-5921; Bett et al. (1994) Proc. Natl. Acad. Sci. USA 91:8802-8806.

Adenovirus vectors are constructed from adenoviruses that have either been isolated from a host or from a laboratory culture. Non-limiting examples of hosts include humans, non-human primates, canines, cows (bovine), and pigs (porcine). Methods of isolating adenoviruses from mammalian hosts are known in the art, see, for example, Darbyshire et al. (1965). J Comp. Pathol. 75:327-330. In general, recombinant adenovirus vectors are made from isolated adenoviruses by insertion of particular elements that improve expression of transgenes into the genome of the adenovirus. Examples of these elements include, but are not limited to, promoters, enhancers, and poly-adenylation signals. See, for example, Carswell and Alwine (1989) Mol. Cell Biol 9(10): 4248-4258 and Huang and Gorman (1990) Nucleic Acids Res. 18(4):937-947.

Construction of a plasmid containing an adenovirus genome is described in Example 1, infra. Nearly full-length adenovirus genomic sequences can be deleted in regions such as E1, E3, E4 and the region between E4 and the right end of the genome. Adenovirus genomes can be deleted in regions essential for replication (i.e., E1 gene) if the essential function can be supplied by a helper cell line. Deletion of essential genes is generally done to increase the size of transgene that can be inserted into the adenovirus genome since there is a size limitation on the amount of genomic material an adenovirus can package. Examples of helper cell lines that constitutively express E1A and E1B genes which can be used to supply essential functions include, but are not limited to, 293 cells, 911 cells, and PER cells for HAV and R2 (Reddy et al., 1999, J Virol. 73:9137-9144; ATCC deposit PTA-156) for BAV. See, for example, Graham et. al. (1977) J Gen. Virol. 36:59-74 and Fallaux et. al. (1998) Hum. Gene Ther. 9:1909-1917.

Insertion of the cloned heterologous sequences into a viral genome occurs by in vivo recombination between a plasmid vector (containing heterologous sequences flanked by adenovirus guide sequences) and an adenovirus genome following co-transfection into a suitable host cell. The adenovirus genome contains inverted terminal repeat (ITR) sequences required for initiation of viral DNA replication and sequences involved in packaging of replicated viral genomes. Reddy et al. (1995) Virology 212:237-239. Adenovirus packaging signals generally lie between the left ITR and the E1A promoter. Incorporation of the cloned heterologous sequences into the adenovirus genome thus places the heterologous sequences into a DNA molecule containing viral replication and packaging signals, allowing generation of multiple copies of a recombinant adenovirus genome that can be packaged into infectious viral particles. Alternatively, incorporation of the cloned heterologous sequences into an adenovirus genome places these sequences into a DNA molecule that can be replicated and packaged in an appropriate helper cell line. Multiple copies of a single sequence can be inserted to improve yield of the heterologous gene product, or multiple heterologous sequences can be inserted so that the recombinant virus is capable of expressing more than one heterologous gene product. Attachment of guide sequences to a heterologous sequence can also be accomplished by ligation in vitro. In this case, a nucleic acid comprising a heterologous sequence flanked by adenovirus guide sequences can be co-introduced into a host cell along with an adenovirus genome, and recombination can occur to generate a recombinant adenovirus vector. Introduction of nucleic acids into cells can be achieved by any method known in the art, including, but not limited to, microinjection, transfection, electroporation, CaPO₄ precipitation, DEAE-dextran, liposomes, particle bombardment, etc.

A recombinant adenovirus expression cassette can be obtained by cleaving a wild-type adenovirus genome with an appropriate restriction enzyme to produce an adenovirus restriction fragment representing, for example, the left end or the right end of the genome comprising E1 or E3 gene region sequences, respectively. The adenovirus restriction fragment can be inserted into a cloning vehicle, such as a plasmid, and thereafter at least one heterologous sequence (which may or may not encode a foreign protein) can be inserted into the E1 or E3 region with or without an operatively-linked eukaryotic transcriptional regulatory sequence. The recombinant expression cassette is contacted with an adenovirus genome and, through homologous recombination or other conventional genetic engineering methods, the desired recombinant is obtained. In the case wherein the expression cassette comprises the E1 region or some other essential region, recombination between the expression cassette and an adenovirus genome can occur within an appropriate helper cell line such as, for example, an E1-transformed cell line. Restriction fragments of the adenovirus genome other than those comprising the E1 or E3 regions are also useful in the practice of the invention and can be inserted into a cloning vehicle such that heterologous sequences can be inserted into the adenovirus sequences. These DNA constructs can then undergo recombination in vitro or in vivo, with an adenovirus genome either before or after transformation or transfection of an appropriate host cell.

In another embodiment of the invention, the kinetics of the adenovirus vectors comprising an intron can also be modulated by the inclusion of different types of promoters within the adenovirus vector construct. The introduction of SV40 early promoter or human cytomegalovirus (HCMV) immediate early (IE) promoter into the expression cassette can change the kinetics of the HE expression, as exemplified in the Examples.

In another aspect of the invention, the present inventors have found that replication-competent (E3 deleted) BAV-3 vector can package up to 3 kb foreign DNA. This is an unexpected result from prior studies which suggests that about 1.5-2kb foreign DNA can be inserted into adenovirus vectors. This finding should prove useful in expression of other RNA viral genes which are larger than 1.5 to 2 kb in size and can be utilized by placing the transgenes larger than 1.5-2 kb in the E3 region of BAV-3 expression system.

V. Uses and administration of adenovirus vectors

The recombinant adenoviruses of the present invention, ie those comprising an intron 5′ to a transgene, can be used to provide protection against a wide variety of diseases affecting cattle, humans and other mammals, such as for example, RNA or DNA viral infection, bacterial infection and/or parasitic infection. Any of the recombinant antigens or immunogens produced or recombinant adenoviruses of the invention can be formulated and used in substantially the same manner as described for antigenic determinant vaccines or live vaccine vectors.

In another aspect of the invention, recombinant adenoviruses of the present invention, ie those comprising an intron 5′ to a transgene, can be used in methods for delivering a gene to a mammal, such as a bovine or a human or other mammal in need thereof, to control a gene deficiency, to provide a therapeutic gene or nucleotide sequence to the host mammal and/or to induce or correct a gene mutation. The method can be used, for example, in the treatment of conditions including, but not limited to hereditary disease, infectious disease, and cardiovascular disease. The method comprises administering to said mammal an adenovirus vector of the present invention comprising an intron and a heterologous transgene wherein said heterologous transgene expresses a desired protein and the intron is inserted upstream of the transgene

In some embodiments, the adenovirus vector genome is incorporated into said mammalian genome or is maintained independently and extrachromosomally to provide expression of the heterologous transgene in the mammalian host. For the purposes of the present invention, the vectors, cells and viral particles prepared by the methods of the invention may be introduced into a subject either ex vivo, (i.e., in a cell or cells removed from the patient) or directly in vivo into the body to be treated.

The present invention also includes pharmaceutical compositions comprising a therapeutically effective amount of a recombinant adenovirus vector, recombinant adenovirus or recombinant protein, prepared according to the methods of the invention, in combination with a pharmaceutically acceptable vehicle and/or an adjuvant or with a buffer. Such a pharmaceutical composition can be prepared and dosages determined according to techniques that are well-known in the art. The pharmaceutical compositions of the invention can be administered by any known administration route including, but not limited to, systemically (for example, intravenously, intratracheally, intraperitoneally, intranasally, parenterally, enterically, intramuscularly, subcutaneously, intratumorally or intracranially) or by aerosolization or intrapulmonary instillation. Administration can take place in a single dose or in doses repeated one or more times after certain time intervals. The appropriate administration route and dosage will vary in accordance with the situation (for example, the individual being treated, the disorder to be treated or the gene or polypeptide of interest), but can be determined by one of skill in the art.

The invention also encompasses methods of treatment and methods for the amelioration of the symptoms associated with a disease or infection by a pathogen, according to which a therapeutically effective amount of an adenovirus vector, recombinant adenovirus, or host cell of the invention is administered to a mammalian subject requiring treatment or in need of amelioration of the symptoms associated with a disease or infection. Amelioration means the prevention, reduction or palliation of a state, such as a symptom associated with disease or infection.

The protein antigens, such as RNA or DNA viral proteins, bacterial proteins or proteins from parasites, used in the present invention, particularly when comprised of short oligopeptides, can be conjugated to a vaccine carrier. Vaccine carriers are well known in the art: for example, bovine serum albumin (BSA), human serum albumin (HSA) and keyhole limpet hemocyanin (KLH). A preferred carrier protein, rotavirus VP6, is disclosed in EPO Pub. No. 0259149, the disclosure of which is incorporated by reference herein.

Genes for desired antigens or coding sequences thereof which can be inserted into adenovirus vectors include those of organisms which cause disease in mammals. For bovine, of particular interest are bovine pathogens such as bovine rotavirus, bovine coronavirus, bovine herpes virus type 1, bovine respiratory syncytial virus, bovine parainfluenza virus type 3 (BPI-3), bovine viral diarrhea virus, Pasteurella haemolytica, Haemophilus sommus, Cryptosporidium and the like. Genes encoding antigens of human pathogens also useful in the practice of the invention. The vaccines of the invention carrying heterologous transgenes or fragments can also be orally administered in a suitable oral carrier, such as in an enteric-coated dosage form. Oral formulations include such normally-employed excipients as, for example, pharmaceutical grades of mannitol, lactose, starch, magnesium stearate, sodium saccharin cellulose, magnesium carbonate, and the like. Oral vaccine compositions may be taken in the form of solutions, suspensions, tablets, pills, capsules, sustained release formulations, or powders, containing from about 10% to about 95% of the active ingredient, preferably about 25% to about 70%. An oral vaccine may be preferable to raise mucosal immunity (which plays an important role in protection against pathogens infecting the gastrointestinal tract) in combination with systemic immunity.

In addition, the vaccine can be formulated into a suppository. For suppositories, the vaccine composition will include traditional binders and carriers, such as polyalkaline glycols or triglycerides. Such suppositories may be formed from mixtures containing the active ingredient in the range of about 0.5% to about 10% (w/w), preferably about 1% to about 2%.

Protocols for administering to animals the vaccine composition(s) of the present invention are within the skill of the art in view of the present disclosure. Those skilled in the art will select a concentration of the vaccine composition in a dose effective to elicit an antibody and/or T-cell mediated immune response to the antigenic fragment. Within wide limits, the dosage is not believed to be critical. Typically, the vaccine composition is administered in a manner which will deliver between about 1 to about 1,000 micrograms of the subunit antigen in a convenient volume of vehicle, e.g., about 1-10 cc. Preferably, the dosage in a single immunization will deliver from about 1 to about 500 micrograms of subunit antigen, more preferably about 5-10 to about 100-200 micrograms (e.g., 5-200 micrograms).

The timing of administration may also be important. For example, a primary inoculation preferably may be followed by subsequent booster inoculations if needed. It may also be preferred, although optional, to administer a second, booster immunization to the animal several weeks to several months after the initial immunization. To insure sustained high levels of protection against disease, it may be helpful to re-administer a booster immunization to the animals at regular intervals, for example once every several years. Alternatively, an initial dose may be administered orally followed by later inoculations, or vice versa. Preferred vaccination protocols can be established through routine vaccination protocol experiments.

The dosage for all routes of administration of in vivo recombinant virus vaccine depends on various factors including, the size of patient, nature of infection against which protection is needed, carrier and the like and can readily be determined by those of skill in the art. By way of non-limiting example, a dosage of between 10³ pfu and 10⁸ pfu and the like can be used. As with in vitro subunit vaccines, additional dosages can be given as determined by the clinical factors involved.

The invention also includes a method for delivering a gene or providing gene therapy to a mammal, such as a bovine or a human or other mammal, in need thereof to control a gene deficiency which comprises administering to said mammal a live recombinant bovine adenovirus containing a foreign nucleotide sequence encoding a non-defective form of said gene under conditions wherein the recombinant virus vector genome is incorporated into said mammalian genome or is maintained independently and extrachromosomally to provide expression of the required gene in the target organ or tissue. These kinds of techniques are currently being used by those of skill in the art to replace a defective gene or portion thereof.

The present inventors have employed the methods and techniques disclosed herein to generate adenovirus vectors demonstrating increased expression of a transgene. Exemplified herein is the expression of a viral protein from bovine herpesvirus. In this embodiment, a replication-competent (E3 deleted) bovine adenovirus-3 (BAV-3) recombinant construct made with the selection of glycoprotein D as the transgene expressed significant amounts of glycoprotein D (gD) of bovine herpesvirus-1 (BHV-1), a DNA virus. In another embodiment of the invention, the present inventors optimized the expression of RNA viral genes. Hemagglutinin esterase (HE) gene of bovine coronavirus (BCV), an RNA virus, was inserted into the E3 region with or without exogenous transcription control elements to generate several BAV-3 recombinant constructs. The introduction of a 137 bp chimeric intron upstream of the HE cDNA was associated with increased HE gene expression. The chimeric intron is composed of the 5′-donor site from the first intron of the human beta-globulin gene and the branch and 3′-acceptor site from the intron of an immunoglobulin gene heavy chain variable region (Senapathy et al., 1990 Meth. Enzymol. 183, 252-278).

The following Examples are provided to illustrate but not limit the present invention.

### EXAMPLES

### Example 1

### Construction of E3 Transfer Vectors

Wild type and recombinant BAV-3 adenovirus were cultivated in Madin Darby bovine kidney (MDBK) and R2 cells, which are transformed fetal bovine retina cells (Reddy et al., 1999 J Virol.73: 9137-9144). The cells were grown in Eagle's minimum essential medium supplemented with to 5% fetal bovine serum. The viral DNA was extracted from virus infected cell monolayers by the method of Hirt (1967 J Mol. Biol. 26, 365-369).

The original E3 transfer vector, pBAV-300, has the genomic DNA sequences between nucleotides (nt) 24465 and 28593 (nt numbers are based on BAV-3 genome sequence; GenBank Accession No. AF030154) with a deletion of 1245 bp of the E3 region from nt 26458 to 27703, cloned into a bacterial plasmid (Zakhartchouk et al., 1998 Virology 250, 220-229). This transfer vector has an overlap of 1992 base pair (bp) on the left side and 889 bp on the right side of the E3 region for homologous recombination in E. coli BJ 5183 having the E3 deleted full-length clone pFBAV-302 (Zakhartchouk et al., 1998 Virology 250, 220-229). To increase the overlap, initially the KpnI-SspI fragment representing the right side of BAV-3 genome between nt 24464 and 34060 was introduced into KpnI and blunt-ended NotI sites of pPOLYII sn 14 (Ladhe et al., 1987 Gene 57: 193-201) to generate plasmid, pBAV-299. The region spanning the Kpnl and XbaI sites of pBAV-299 was replaced with that of pBAV-300 to generate pBAV-301. The plasmid pBAV-301 was digested with KpnI (nt 24464) and Spel (nt 31570) enzymes, subjected to gel electrophoresis, and the gel purified fragment was used for homologous recombination in E. coli BJ 5183. This new transfer vector has two unique restriction enzyme sites (SrfI and SaII) for cloning of foreign genes and an overlap of 1992 bp on the left side and 3866 bp on the right side of the E3 region for efficient homologous recombination with plasmid pFBAV-302 (Zakhartchouk et al., 1998 Virology 250, 220-229), which dramatically increased the frequency of recombination in BJ 5183 cells.

The plasmid pBAV301b was constructed by cloning a 137 bp long chimeric intron amplified by PCR from pCI-neo (Promega) into the SrfI site of pBAV-301. The intron is composed of the 5′-donor site from the first intron of the human beta-globulin gene and the branch and 3′-acceptor site from the intron of an immunoglobulin gene heavy chain variable region (Senapathy et al., 1990 Meth. Enzymol. 183, 252-278). The transgenes are introduced downstream of the intron in order to prevent utilization of possible cryptic 5′-donor splice sites, that is nucleic acid sequences present in the transgene that are susceptible to splicing events, within the BCV HE cDNA sequence.

### Example 2

### Construction of Recombinant Plasmids

a) Construction of plasmid pFBAV303 and pFBAV332

The 1.3 kb BamHI fragment of a plasmid pCVE3 (Parker et al., 1989 J. Gen. Virol. 70, 155-164) containing the complete coding sequence of BCV HE gene was treated with T4 DNA polymerase and ligated to blunt end repaired SrfI digested plasmid pBAV301 to create plasmid pBAV301.HE and blunt end repaired SalI digested plasmid pBAV301b to create plasmid pBAV301b.HE. The recombinant BAV-3 genomes containing the gene encoding HE were generated by homologous recombination in E. coli BJ5183 between SrfI linearized pFBAV302 and 7.2 kb KpnI-SpeI fragment of pBAV301.HE creating plasmid pFBAV303, and between SrfI linearized pFBAV302 and 7.3 kb KpnI-SpeI fragment of pBAV301b.HE creating plasmid pFBAV332.

b) Construction of Plasmid pFBAV333 and pFBAV334

The plasmid pSVPIA containing a unique SalI cloning site was constructed by ligating 209 bp of SV40 promoter (isolated from pCAT-Promoter plasmid; Promega), 137 bp chimeric intron and 240 bp SV40 late poly (A) signal (isolated from pCI-neo; Promega) to plasmid pPOLYIIsn. The plasmid pCMVPIA is similar to plasmid pSVPIA except that the SV40 promoter is replaced by 510 bp HCMV promoter (isolated from pCMVβ; Clontech). The 1.3 kb blunt end repaired BamHI fragment containing HE gene (Parker et al., 1989 J Gen. Virol. 70, 155-164) was ligated to blunt end repaired SalI digested plasmid pSVPIA to create plasmid pSVPIA.HE, and to blunt end repaired SalI digested plasmid pCMVIA to create plasmid pCMVIA.HE. A 1.9 kb fragment of plasmid pSVPIA.HE and a 2.0 kb fragment of plasmid pCMVPIA.HE containing the HE gene under appropriate transcriptional elements were isolated and ligated individually to blunt end repaired SrfI digested plasmid pBAV301 to create plasmids pBAV30I.HEsv and pBAV302.HEcmv, respectively. Finally, the recombinant BAV-3 genomes were isolated by homologous recombination in E. coli BJ5183 between SrfI linearized plasmid pFBAV302 and 7.8 kb KpnI-SpeI fragment of pBAV301.Hesv creating plasmid pFBAV333, and between SrfI linearized plasmid pFBAV302 and 8.1 kb KpnI-SpeI fragment of pBAV301.HEcmv creating plasmid pFBAV334.

c) Construction of Plasmid pFBAV335, pFBAV336 and pFBAV337

The full length gB gene of bovine herpesvirus-1 (BHV-1), excised from plasmid pSLIAgB as a 2903 bp BgII fragment, was blunt end repaired and cloned into the SrfI site of pBAV301 creating plasmid pBAV301.gB. Homologous recombination in BJ5183 between 8.8 kb KpnI-SpeI fragment of pBAV301.gB and SrfI linearized pFBAV302 created plasmid pFBAV335. The truncated gB gene of BHV-1, excised from plasmid pSLIAtgB as a 3020 bp BamHI-KpnI fragment, was blunt end repaired and cloned into the SrfI site of pBAV301 creating plasmid pBAV301.tgB. Homologous recombination between 8.9 kb Kpn-SpeI fragment of pBAV301.tgB and SrfI linearized pFBAV302 created plasmid pFBAV336. The full length LacZ gene, excised from plasmid pCMVβ as 3246 bp SmaI-DraI fragment was cloned into blunt end repaired SalI site of pBAV301b creating plasmid pBAV301b.LacZ. Homologous recombination between 9.3 kb KpnI-SpeI fragment of pBAV301b.LacZ and SrfI linearized pFBAV302 created plasmid pFBAV337.

### Example 3

### Growth, Isolation, and Characterization of Recombinant BAV-3

R2 cell monolayers (transformed fetal bovine retina cells) in 60 mm dishes were transfected with 5-10 μg of PacI digested pFBAV303, pFBAV332, pFBAV333, pFBAV334, pFBAV335, pFBAV336 and pFBAV337 recombinant plasmid DNAs using lipofectin. After incubation at 37° C., the transfected cells showing cytopathic effects were collected, freeze-thawed two times and the recombinant viruses were plaque purified on MDBK cells.

Southern blot hybridization

DNA fragments obtained after restriction enzyme digestion of virion DNA were transferred from agarose gels to Nytran membranes (Schleicher and Schuell) as described (Reddy et al., 1993 Intervirol. 36, 161-168). The gene coding for HE of BCV or gB of BHV-1 was labeled with ³²p dCTP by the random primer labeling technique (Sambrooke 1989 supra). Hybridizations were carried out at 42° C. in the presence of 50% formamide. Prehybridization, hybridizations and washing of membranes were carried out as described in (Reddy et al., 1993 Intervirol. 36, 161-168).

Northern Blot Hybridizations

MDBK cells grown in petri dishes were infected with 5 plaque forming units (pfu) per cell of recombinant BAV-3s. Total RNA was extracted from mock infected or recombinant BAV-3 infected cells with acid guanidinium thiocyanate-phenol-chloroform mixture as described by Chomczynski & Sacchi (1987 Anal. Biochem. 162, 156-159). RNA (10 μg) was separated on 1% agarose-formaldehyde gels and transferred to Nytran membranes. The blots were baked, prehybridized, hybridized and washed as described. The gene coding for HE of BCV was labeled with α³²P dCTP by the random primer labeling technique (Sambrooke, 1989 supra) and used as a probe.

Immunoprecipitations

Confluent monolayers of MDBK cells in 6 well dishes were infected with the virus at a multiplicity of infection of least five. The cells were preincubated for 2 hours in MEM deficient in methionine and cystine prior to labeling with 50 μci of ³⁵S methionine (Trans (³⁵S) label [1,000 Ci/mmol] ICN Radiochemicals Inc., Irvine, Calif.) for 4h. The cells were washed once with PBS, harvested by scraping and then lysed with ice-cold modified radioimmunoprecipitation assay buffer. The radiolabeled proteins were immunoprecipitated with polyclonal anti-BCV rabbit antibodies (Deregt & Babiuk, 1987 Virology 161, 410-420) or monoclonal anti-gB antibodies (van Drunen Littel-van den Hurk et al., 1984 Virology 135, 466-479) and analyzed on a SDS-polyacrylamide gel electrophoresis (PAGE) under reducing conditions. The gels were dried and protein bands were visualized by autoradiography.

### Example 4

### Generation and Characterization of Recombinant BAVs Containing the HE Gene of BCV

Initial attempts to insert the BCV HE gene in E3 region of plasmid pFBAV302 (E3 deleted full length BAV-3 genomic clone; Zakhartchouk et al, 1998 Virology 250, 220-229) by homologous recombination in E. coli BJ5183 between KpnI--XbaI fragment (with an overlap of 1992 bp on left side and 889 bp on the right side of E3 region) of plasmid pBAV300 (Zakhartchouk et al., 1998 Virology 250, 220-229) containing BCV HE gene and SrfI linearized plasmid pFBAV302 were unsuccessful. In order to increase the efficiency of inserting foreign genes in the E3 region of plasmid pFBAV302 by homologous recombination in E coli BJ5183, we first constructed a modified transfer plasmid pBAV301. This plasmid contains an overlap of 1992 bp on the left side and 3866 bp on the right side of the E3 region with plasmid pFBAV302. Use of this plasmid dramatically increased the frequency of recombination in E. coli BJ5183 and BCV HE was successfully cloned.

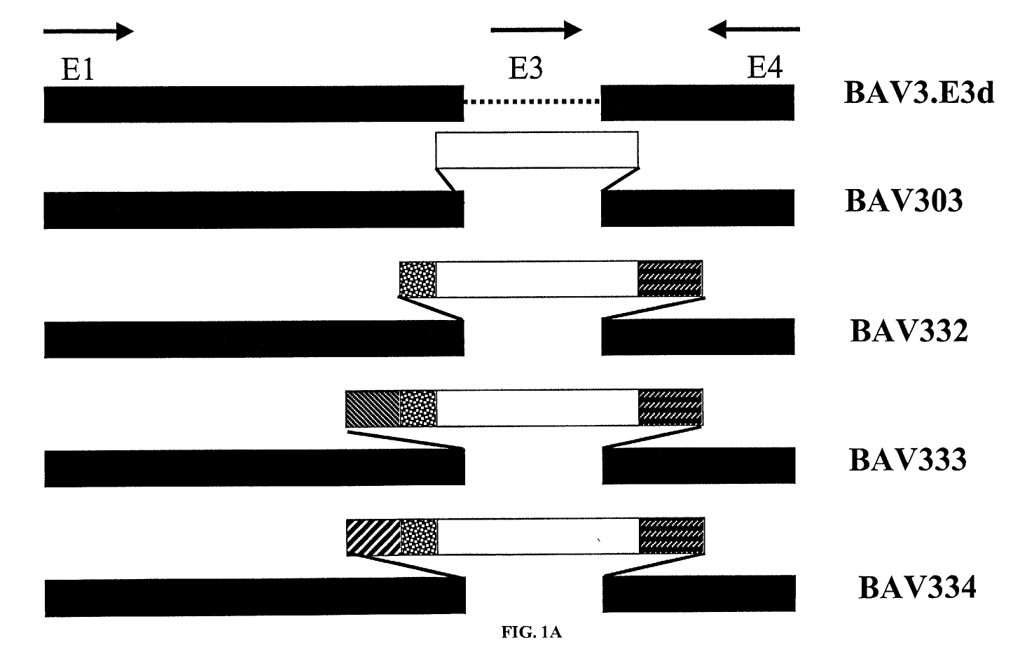

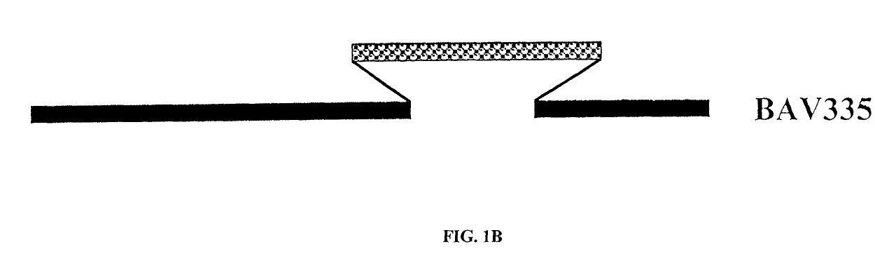

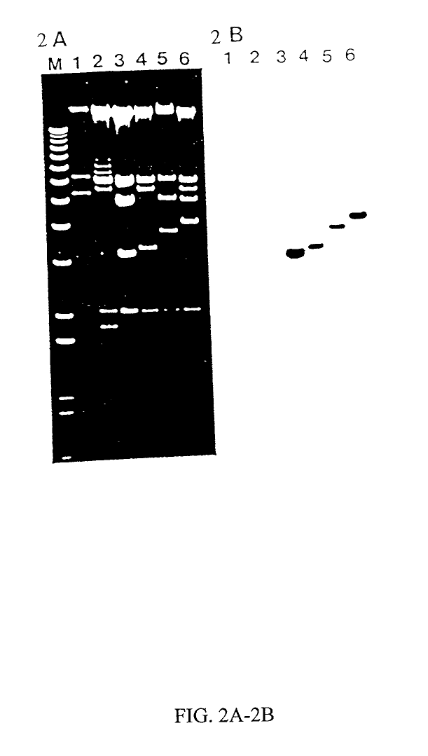

Next, recombinant BAV-3 s expressing the BCV glycoprotein HE were constructed. The full length HE gene alone or with different exogenous transcriptional elements was inserted individually into the E3 region of plasmid pFBAV302 in the same transcriptional orientation of E3 using the homologous recombination machinery of E. coli (Chartier et al., 1996 J Virol. 70, 4805-4810). The Pac-I digested pFBAV303, pFBAV332, pFBAV333 or pFBAV334 plasmid DNA was transfected into R2 cells (transformed fetal bovine retina cells). The infected monolayers showing 50% cytopathic effects were collected, freeze thawed and recombinant viruses were plaque purified and propagated in MDBK cells. The recombinant viruses were named BAV303 (HE without exogenous elements), BAV332 (HE with chimeric intron), BAV333 (HE with SV40 promoter chimeric intron and SV40 polyA) and BAV334 (HE with CMV promoter, chimeric intron, SV40 polyA) (FIG. 1). The viral DNA was extracted from infected cells by Hirt method (Hirt, 1967 J Mol. Biol. 26, 365-369) and analysed by agarose gel electrophoresis after digestion with BamHI restriction enzyme. The digestion of wild type BAV-3 viral DNA with BamHI yielded 5 fragments and the fragment D (3.019 kb) contains the E3 region (FIG. 2A, lane 1). The difference in the size of modified BamHI “D” fragment of BAV3.E3 d (FIG. 2A, lane 2) BAV303 (FIG. 2A, lane 3), BAV332 (FIG. 2A, lane 4), BAV333 (FIG. 2A, lane 5) and BAV334 (FIG. 2A, lane 6) was as expected. This was confirmed by Southern blot analysis of BamHI digested genomic DNA of wild type and recombinant BAV3s. As seen in FIG. 2B, the same modified BamHI “D” fragment(s) from the recombinant viruses hybridized to a-³²P dCTP labeled HE gene in the Southern blot hybridization (FIG. 2B, lanes 3,4,5,6). This suggested that recombinant BAV303, BAV332, BAV333 and BAV334 contained the BCV HE gene.

Northern Analysis of HE Transcripts

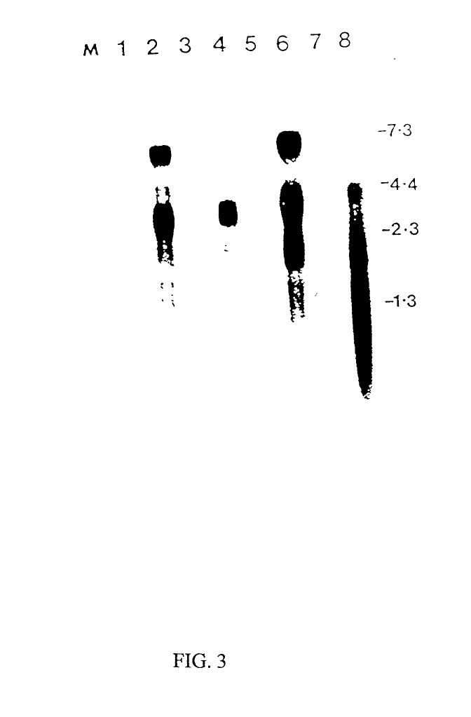

To analyze transcription of the HE gene, RNA was prepared from mock infected or recombinant BAV-3 infected cells at 18 and 28 h post infection. The RNA was separated on agarose-formaldehyde gels, transferred to Nytran membrane and probed with the ∝³²p labeled HE gene. The probe was expected to detect four L6 mRNAs (100K, 33K, 23K and pVIII) and the mRNAs of HE transcribed from the E3 promoter and MLP. Unlike in HAV-2 (Ziff & Fraser, 1978 J Virol. 25, 897-906), the transcripts from the L6 region in BAV-3 were polyadenylated at the poly(A) site of the E3 region (Reddy et al., 1998 J Virol. 72, 1394-1402). Thus all the transcripts of L6 region originating from the major late promoter (MLP) formed a nested set of overlapping molecules with common 3′ ends. Each mRNA contained all the nucleotide sequences in the next smaller mRNA plus one additional ORF at the 5′ end. Only the ORF at the 5′ end of the mRNA was translated. When RNA was analyzed, several abundant mRNAs were identified that had HE sequences in them particularly in RNA that was extracted at 28 h post infection (FIG. 3). At 28 h post infection, the larger transcripts were the dominant species in RNA extracted from BAV302 (lane 2) and BAV334 (lane 6) infected cells. During early stages of HAV-5 infection, the E3 promoter was used to express mRNAs from the E3 region and during late stages, transcription from the E3 promoter was reduced and some mRNAs were made from the MLP (Tollefson et al., 1992 J Virol. 66, 3633-3642). The major late E3 mRNAs containing tripartite leader sequences were also produced in BAV-3 (Idamakanti et al., 1999 Virology 256, 351-359). The sizes of the transcripts were considerably larger than the genomic distance between the E3 promoter and poly(A) site of the E3 region. These transcripts must have been generated by splicing of the primary transcripts produced from the MLP.

### Example 5

### Kinetics of HE Expression in MDBK Cells

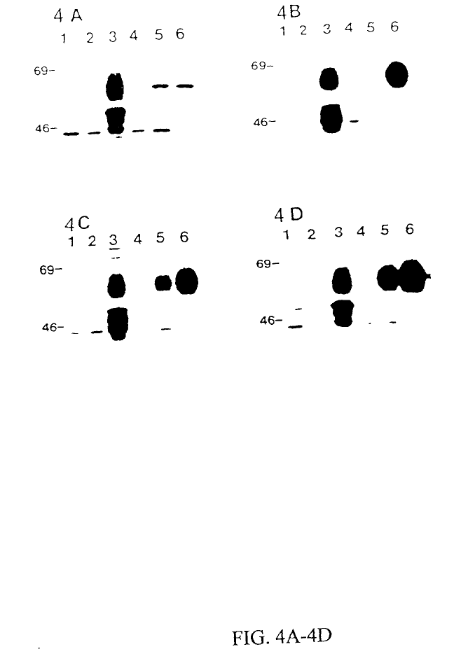

Proteins from cells lysates, collected at different times post infection of MDBK cells with recombinant BAV3, were analyzed by immunoprecipitation assays using BCV specific polyclonal antiserum. Electrophoretical analysis of metabolically radiolabeled immunoprecipitates from BCV (FIG. 4ABCD, lane 3) infected cell lysates detected a protein of 65 kDa. No such protein was detected from mock (FIG. 4ABCD, lane 1) or BAV-3 (FIG. 4ABCD, lane 2) infected cell lysates. The recombinant BAV303 contained the HE cDNA sequence substituting for BAV-3 E3 in parallel orientation so as to allow expression from endogenous promoters. Immunoprecipitation analysis of BAV303 infected cell lysates showed little or no HE expression (FIG. 4A, lanes 4,5,6). The recombinant BAV332 comprises the HE sequence in the E3, downstream of an exogenous chimeric intron and upstream of SV40 late poly(A) signal. Immunoprecipitation analysis of BAV332 infected cell lysates detected a specific band of 65 kDa at 36 hrs post infection (FIG. 4B, lane 6). The recombinant BAV333 and recombinant BAV334 were similar to BAV332 except that they had either SV40 or CMV immediate early promoters upstream of chimeric introns, respectively. Immunoprecipitation analysis of BAV333 infected cells detected a specific band of 65 kDa at 24 (FIG. 4C, lane 5) and 36 (FIG. 4C, lane 6) hrs post infection. Similarly, immunoprecipitation analysis of BAV334 infected cells also detected a specific band of 65 kDa at 24 (FIG. 4D, lane 5) and 36 (FIG. 4D, lane 6) hrs post infection.

The BCV HE gene under the control of SV40 promoter was cloned and expressed using HAV-5 (Yoo et al., 1992 J Gen. Virol. 73, 2591-2600). Expression of the HE was seen as early as 6 h post infection and produced throughout the infection. However, BAV-3 differs from HAV-5 with respect to replication kinetics. In BAV-3 infected cells, viral DNA replication begins at about 24 h post infection and reaches a peak after 40 h, whereas viral DNA replication in HAV-5 infected cells occurs as early as 12 h post infection (Niiyama et al., 1975 J Virol 16, 621-633). The green fluorescent protein (GFP) expression was noticed at 12 h post infection when the gene for GFP was placed under the control of CMV immediate early promoter in the E3 ) region of BAV-3 (Reddy et al., 1999 J Virol. 73, 9137-9144). This suggests that kinetics of foreign gene expression from the E3 region of BAV-3 may be influenced not only by exogenous transcriptional elements but also by the nature of the foreign gene.

### Example 6 Determination of Packaging Capacity of E3 Deleted Vectors

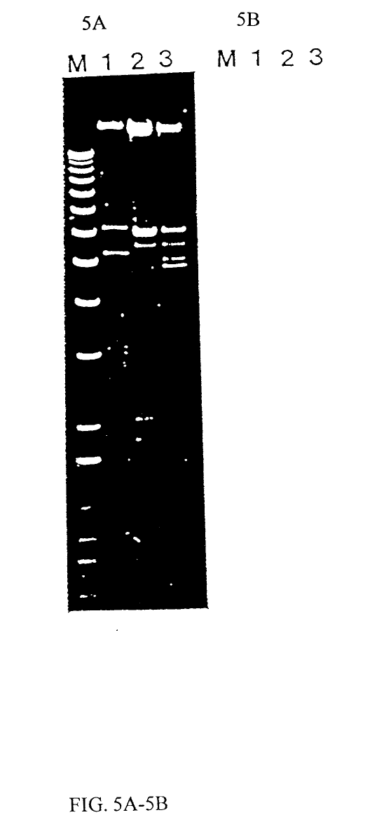

In order to determine the packaging capacity of BAV3.E3 d genome, we constructed E3 deleted BAV-3 full length genomic clones named pFBAV335, pFBAV336 and pFBAV337 containing 2903 bp, 3020 bp or 3246 bp foreign DNA respectively. In each case, the orientation of the insert was the same as the E3 transcription unit. Two clones for each of plasmids pFBAV335, pFBAV336 or pFBAV337 were digested with PacI and transfected into R2 cells. Only cells transfected with plasmid pFBAV335 DNA produced cytopathic effects. The recombinant virus named BAV335 (FIG. 1) was propagated on MDBK cells. The DNA extracted from recombinant virus infected cells was analyzed by restriction enzyme digestions (FIG. 5A). As expected, the BamHI “D” fragment of BAV-3 is 3.019 kb (lane 1), BAV3.E3d is 1.8 kb (lane 2) and BAV335 is 4.6 kb (lane 3). This was confirmed by Southern blot analysis of BamHI digested genomic DNA of wild type and recombinant BAV-3s. As seen in FIG. 5B, the same modified BamHI “D” fragment from recombinant BAV335 hybridized to α-³²P dCTP labeled gB gene in the Southern blot hybridization (FIG. 5B lane 3). This confirmed that recombinant BAV335 contained the gB gene.

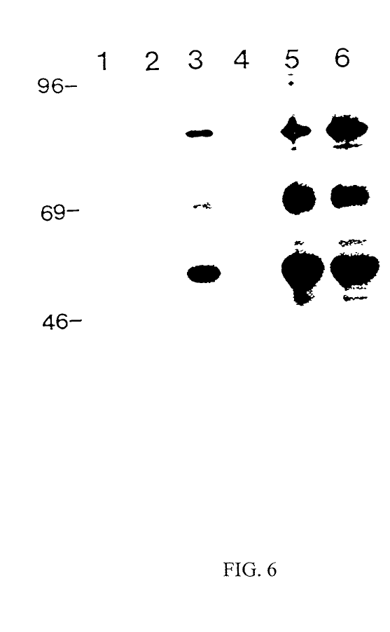

In order to determine the expression of the gB protein, recombinant BAV335 virus infected radiolabeled cell lysates were immunoprecipitated with a pool of gB specific MAbs (van Drunen Littel-van den Hurk et al., 1984 Virology 135, 466-479) and analysed by SDS-PAGE under reducing conditions. As seen in FIG. 6, immunoprecipitation of recombinant BAV335 infected cells revealed three bands of 130, 74 and 55 kDa from BAV335 infected cells (lanes 5,6), which co-migrated with gB produced in BHV-1 infected cells (lane 3). No similar bands were observed in uninfected cells (lane 1), or cells infected with recombinant BAV3.E3d (lane 2). The recombinant gB was expressed at 24 hours (lane 5) and 36 hours (lane 6) but not at 12 hours (lane 4) post infection.

One of the most important characteristics of any viral vector is the packaging capacity. As any other icosahedral viral vector, adenoviruses also have a limited vector capacity. Adenovirus capsids can package genomes as big as 105% of the size of the wild-type genome (Ghosh-choudhury et al., 1987 EMBO J 6,1733-1739), which allows for insertion of approximately 1.8 to 2.0 kb of excess DNA. To clone foreign genes larger than 2.0 kb in adenovirus vectors, compensatory deletions are usually made in the E1 and E3 regions of the genome. However, recently it was reported that the packaging capacity of a ovine adenovirus vector exceeds 5% packaging rule (Xu et al., 1997 Virology 230, 62-71). To determine the packaging capacity of E3 deleted BAV-3 vector, genes coding for authentic gB (2903 bp), truncated gB (3020 bp) of BHV-1 and β galactosidase (3246 bp) were introduced into the E3 region of full length plasmid, pFBAV-302 (Zakhartchouk et al., 1998 Virology 250, 220-229). A virus could only be rescued from the full-length plasmid containing authentic gB gene indicating that the vector capacity of BAV-3 is about 2.9 kb. According to 5% packaging rule of HAV-5 (Ghosh-choudhury et al., 1997), the theoretical packaging capacity of E3 deleted BAV-3 (Zakhartchouk et al., 1998 Virology 250, 220-229), is similar to the packaging capacity determined in this study.

### Example 7

### Growth of Recombinant Viruses

To determine whether insertion of exogenous transcription control elements into the E3 had any noticeable effect on the ability of these recombinant to replicate in MDBK cells, virus titers were determined. Deletion of the E3 region had no detectable effect on the virus yield. BAV303, BAV332, BAV333 and BAV335 grew to similar titers as BAV3.E3d (E3 deleted) virus. However, BAV334 grew to a final titer that was 1.0 log10 lower than the BAV3.E3d (E3 region deleted virus, Zakhartchouk et al., 1998 Virology 250, 220-229).

What is claimed is:

1. An adenovirus vector comprising an intron and a heterologous transgene wherein said intron is located 5′ to the heterologous transgene, and wherein said vector is capable of expressing greater levels of the heterologous transgene than a comparable adenovirus vector comprising a heterologous transgene and lacking an intron 5′ to said heterologous transgene.

2. The adenovirus vector of claim 1, wherein said vector is mammalian or avian.

3. The adenovirus vector of claim 2, wherein mammalian includes human, non-human primate, bovine, porcine, canine, or ovine.

4. The adenovirus vector of claim 2, wherein said vector is bovine adenovirus vector.

5. The adenovirus vector of claim 4, wherein said bovine adenovirus vector is a member of subgroup 1 bovine adenovirus or subgroup 2 bovine adenovirus.

6. The adenovirus vector of claim 4, wherein said bovine adenovirus vector is BAV3.

7. The adenovirus vector of claim 1, wherein said transgene encodes a eucaryotic or procaryotic protein.

8. The adenovirus vector of claim 7 wherein said transgene encodes a therapeutic protein or polypeptide; a growth hormone or other growth enhancer; or a protein capable of eliciting an immune response.

9. The adenovirus vector of claim 7, wherein said transgene encodes a protein from a pathogen.

10. The adenovirus vector of claim 9, wherein said protein is an RNA viral protein.

11. The adenovirus vector of claim 9 wherein said protein is a DNA viral protein.

12. The adenovirus vector of claim 9, wherein said protein is a bacterial protein.

13. The adenovirus vector of claim 9, wherein said protein is a protein from a parasite.

14. The adenovirus vector of claim 1, wherein said intron is a mammalian intron.

15. The adenovirus vector of claim 1, wherein said transgene is operably linked to a control region and said intron is located 3′ to said control region.

16. The adenovirus vector of claim 1, wherein said vector is replication-competent.

17. The adenovirus vector of claim 1, wherein said vector is replication-defective.

18. A composition comprising a vector according to claim 1.

19. The composition of claim 18 further comprising a pharmaceutically acceptable excipient.

20. A host cell comprising the vector of claim 1.

21. A recombinant adenovirus comprising the vector of claim 1.

22. A method of preparing an adenovirus vector comprising an intron and a heterologous transgene wherein said intron is located 5′ to said heterologous transgene, said method comprising the steps of obtaining an adenovirus vector and inserting a transgene and an intron into said vector, wherein said intron is inserted 5′ to said heterologous transgene.

23. The method of claim 22 wherein said adenovirus vector has a deletion in a gene essential for replication.

24. The method of claim 23 wherein said gene essential for replication is E1.

25. A method of preparing an adenovirus comprising the adenovirus vector of claim 1, comprising the steps of culturing a mammalian host cell comprising the adenovirus vector of claim 1 under conditions suitable for adenovirus replication and packaging; and
optionally recovering said adenovirus produced.

26. The method according to claim 25 wherein said adenovirus has a deletion in a gene essential for replication and said method further comprises the step of culturing said mammalian host cell in the presence of a helper cell line which comprises said gene essential for replication.

27. The method of claim 26 wherein said gene essential for replication is E1.

28. An immunogenic composition comprising an adenovirus vector of claim 9.

29. An immunogenic composition comprising an adenovirus vector of claim 10.

30. An immunogenic composition comprising an adenovirus vector of claim 11.

31. An immunogenic composition comprising an adenovirus vector of claim 12.

32. An immunogenic composition comprising an adenovirus vector of claim 13.

33. A composition capable of inducing an immune response in a mammalian subject, said composition comprising the immunogenic composition of claim 28.

34. The composition according to claim 33 further comprising a pharmaceutically acceptable excipient.

35. A method of treating or ameliorating the symptoms of a RNA viral infection in a mammalian host comprising administering to said host a therapeutically effective amount of the immunogenic composition of claim 29.

36. A method of treating or ameliorating the symptoms of a DNA viral infection in a mammalian host comprising administering to said host a therapeutically effective amount of the immunogenic composition of claim 30.

37. A method of treating or ameliorating the symptoms of a bacterial infection in a mammalian host comprising administering to said host a therapeutically effective amount of the immunogenic composition of claim 31.

38. A method of treating or ameliorating the symptoms of a parasitic infection in a mammalian host comprising administering to said host a therapeutically effective amount of the immunogenic composition of claim 32.
